(19) United States
(12) Patent Application Publication
(10) Pub. No.: US 2004/0007254 A1
(43) Pub. Date: Jan. 15, 2004

(54) Cleaning tool

(76) Inventors: Laust Pedersen, Santa Barbara, CA (US); Charles Pitman, Santa Barbara, CA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/194,200

(22) Filed: Jul. 12, 2002

(51) Int. Cl. B08B 9/00

(52) U.S. Cl. 134/22.1; 134/22.18; 134/166 R; 134/171

(57) ABSTRACT

The invention features cleaning tools for brachytherapy applicators. In particular, the invention features devices that are useful for flushing areas within and adjacent to a magazine receiving well of a brachytherapy applicator in order to remove debris and contaminants. Such tools can be used to restore the function of magazine retaining structures of brachytherapy applicators.

### TECHNICAL FIELD

This invention relates to devices for cleaning brachytherapy applicators.

### BACKGROUND

Brachytherapy is a form of cancer treatment in which radiation sources are placed inside a patient's body to irradiate a tumor. In brachytherapy, a surgeon uses an applicator to implant radioactive seeds in or around a tumor, allowing localized and precise irradiation of the tumor. Many forms of cancer respond to brachytherapy, including several forms of prostate cancer. Brachytherapy generally is less invasive than surgery, usually results in fewer side effects than surgery or external beam radiation, allows for a shorter recovery time, and reduces the impact on the patient's quality of life. Brachytherapy applicators typically contain a long needle that can be inserted into a tumor (e.g., a prostate tumor) so that radioactive seeds can be passed into the tumor.

### SUMMARY

The invention features cleaning tools for brachytherapy applicators. In particular, the invention features devices that are useful for flushing areas within and adjacent to a magazine receiving well of a brachytherapy applicator in order to remove debris and contaminants.

The cleaning tools provided herein contain a first member with an inlet and a second member with one or more outlets, with the first member being connected to the second member such that the inlet and the outlet are fluidly connected. The cleaning devices can be inserted into the magazine receiving well of a brachytherapy applicator. Cleaning fluid that is then forced into the inlet can exit the cleaning device through the one or more outlets. The fluid can be directed into areas within and adjacent to the magazine receiving well, in a manner that is determined by the positioning of the one or more outlets. Cleaning devices of the invention are particularly useful for removing blood cells and other debris from a magazine receiving well and areas around a magazine receiving well. In some embodiments, the cleaning tool includes a structure for actuating a magazine retaining structure within the magazine receiving well. Such a structure can permit cleaning fluid to access the magazine retaining structure and prevent or alleviate potential freezing of the structure due to accumulated debris.

The invention is based on the discovery that a cleaning tool having an inlet fluidly connected to an outlet can be inserted into the magazine receiving well of a brachytherapy applicator, and fluid can be forced through the device such that the magazine receiving well, a magazine retaining structure within the well, and/or areas outside the well can be effectively flushed of debris and contaminants such as blood cells. The invention also is based on the discovery that while blood cells and other contaminants can accumulate in a brachytherapy applicator and can prevent the actuation of a magazine retaining structure, cleaning tools can be designed to actuate the magazine retaining structure and permit cleaning fluid to access the magazine retaining structure, thus restoring actuation capability.

The invention features a device for cleaning a brachytherapy applicator. The device contains: (a) a first member defining an inlet; and (b) a second member configured to fit into a magazine receiving well of the brachytherapy applicator. The second member can define at least three outlets, and the first and second members can be connected such that the inlet is fluidly connected to the at least three outlets. The first member can be configured such that a syringe fits into the inlet, or such that the inlet is connectable to tubing. The inlet can be circular with a diameter of at least about one millimeter. The second member can define a bottom surface and a side surface, and the side surface can define a recess. The recess can extend vertically from the bottom surface for a distance greater than about two millimeters. The depth of the recess can be between about 0.3 millimeter and about one millimeter. An outlet can be positioned in the recess. The second member can fit into the magazine receiving well with a clearance between about 0.01 millimeter and about three millimeters. The second member can define a channel. The second member can define two parallel side surfaces, and the channel can extend between the two parallel side surfaces. A stylet of the brachytherapy applicator can fit through the channel such that the device is aligned within the magazine receiving well. The stylet can fit through the channel with a clearance between about 0.01 millimeter and about three millimeters. The outlet can be circular with a diameter of at least about 0.1 millimeter. The second member can define at least five outlets fluidly connected to the inlet. The first member can have a width between about 0.5 centimeter and about two centimeters. The device can have a length between about four centimeters and about six centimeters. The device can contain plastic or metal.

The invention also features a device for cleaning a brachytherapy applicator. The device can include: (a) a first member defining an inlet; (b) a second member configured to fit into a magazine receiving well of the brachytherapy applicator, wherein the second member defines an outlet, and wherein the first and second members are connected such that the inlet is fluidly connected to the outlet; and (c) a pusher configured to apply force to a magazine retaining structure of the brachytherapy applicator, wherein the pusher is positioned adjacent to the outlet. The first member can be configured such that a syringe fits into the inlet, or such that the inlet is connectable to tubing. The inlet can be circular with a diameter of at least about one millimeter. The second member can define a bottom surface and a side surface, and the side surface can define a recess. The recess can extend vertically from the bottom surface for a distance greater than about two millimeters. The depth of the recess can be between about 0.3 millimeter and about one millimeter. An outlet can be positioned in the recess.

The second member can fit into the magazine receiving well with a clearance between about 0.01 millimeter and about three millimeters. The second member can define a channel. The second member can define two parallel side surfaces, and the channel can extend between the two parallel side surfaces. A stylet of the brachytherapy applicator can fit through the channel such that the device is aligned within the magazine receiving well. The stylet can fit through the channel with a clearance between about 0.01 millimeter and about three millimeters. The outlet can be circular with a diameter of at least about 0.1 millimeter. The second member can define more than one outlet (e.g., five outlets) fluidly connected to the inlet. The pusher can contain an arm and a base. The base can be attached to the arm at an angle, and the base can define a protrusion. The angle can be less than 180 degrees and greater than 135 degrees. The height of the protrusion can be between about 0.3 millimeter and about one millimeter. The protrusion can be positioned within about 3 millimeters of the outlet. The base can have a rounded pivot surface. The first or second member can define a groove, and the rounded pivot surface can engage the groove. The pusher can pivot about the rounded pivot surface. The pusher can have a lever ratio between about 1/1 and about 10/1 (e.g., between about 3/1 and about 5/1). The first or second member can contain at least one attachment point, and the pusher can engage the at least one attachment point. The at least one attachment point can be a flexible clip. The first member can have a width between about 0.5 centimeter and about two centimeters. The device can have a length between about four centimeters and about six centimeters. The device can contain plastic or metal. The magazine retaining structure can be a ball-plunger, and the force can move the ball of the ball-plunger in a direction toward the interior of the ball-plunger.

In another aspect, the invention features a method for cleaning a brachytherapy applicator. The method can involve: (a) providing the brachytherapy applicator, wherein the brachytherapy applicator contains a magazine receiving well and a magazine retaining structure; (b) providing a device for cleaning the brachytherapy applicator; (c) inserting the second member into the magazine receiving well; and (d) inserting fluid into the inlet such that the fluid exits the outlet, thereby cleaning the brachytherapy applicator. The device for cleaning the brachytherapy applicator can contain: (i) a first member defining an inlet; (ii) a second member configured to fit into the magazine receiving well, wherein the second member defines an outlet, and wherein the first and second members are connected such that the inlet is fluidly connected to the outlet; and (iii) a pusher configured to apply force to a the magazine retaining structure, wherein the pusher is positioned adjacent to the outlet.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a side view of a brachytherapy applicator cleaning tool.

FIG. 2 is an opposite side view of the cleaning tool of FIG. 1.

FIG. 3 is a cross-sectional view of the cleaning tool of FIG. 1.

FIG. 4 is an underside view of a brachytherapy applicator cleaning tool having a pusher.

FIG. 5 is an overhead view of the base of the cleaning tool of FIG. 4.

FIG. 6 is a side view of the second member of the cleaning tool of FIG. 4.

FIG. 7 is an end view of the second member of the cleaning tool of FIG. 4.

FIG. 8 is an overhead view of the pusher of FIG. 4.

Like reference symbols in the various drawings indicate like elements.

### DETAILED DESCRIPTION

The invention features cleaning tools for brachytherapy applicators. Specifically, the invention features devices that are useful for flushing areas within and adjacent to a magazine receiving well of a brachytherapy applicator in order to remove debris and contaminants. The cleaning tools provided herein can have any suitable size, provided that at least one portion of the tool can fit into a magazine receiving well of a brachytherapy applicator. A cleaning tool can be, for example, between about one centimeter and about two centimeters wide (e.g., one centimeter, 1.2 centimeters, 1.5 centimeters, 1.7 centimeters, or two centimeters wide), about four centimeters to about six centimeters long (e.g., four centimeters, 4.2 centimeters, 4.5 centimeters, 4.75 centimeters, five centimeters, 5.3 centimeters, 5.6 centimeters, 5.8 centimeters, or six centimeters long), and about 0.5 centimeters to 1.5 centimeters deep (e.g., 0.5 centimeter, 0.75 centimeter, one centimeter, 1.25 centimeters, or 1.5 centimeters deep). Further, the tools provided herein can be made of any suitable material or combination of suitable materials (e.g., metal, plastic, or a combination of metal and plastic).

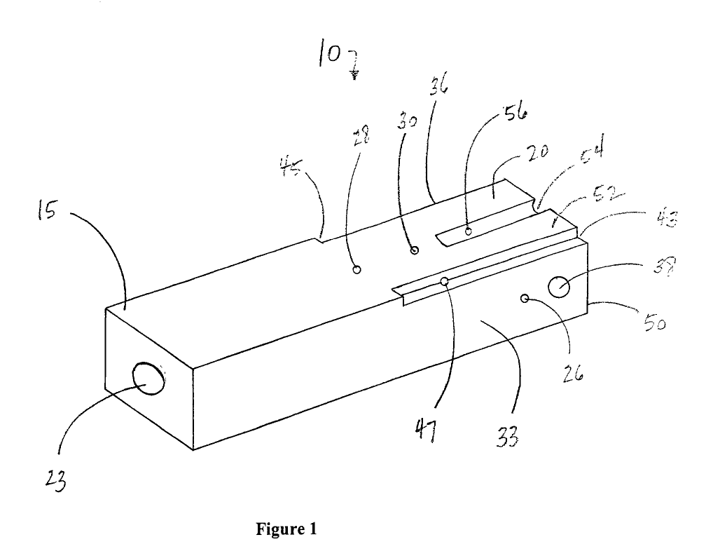

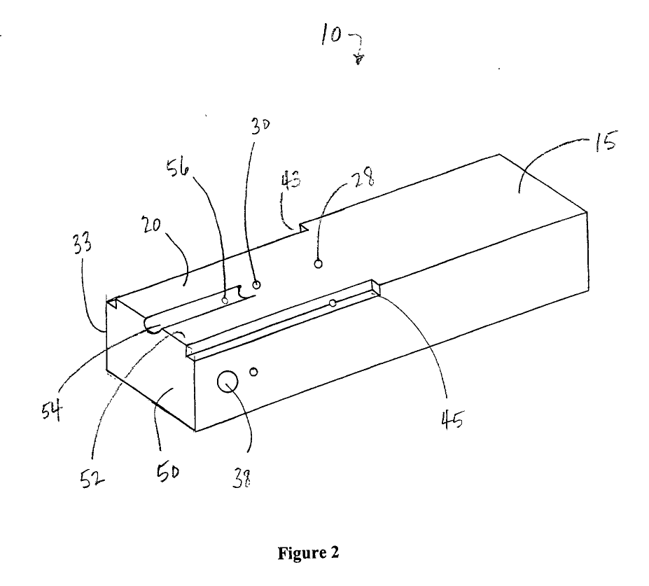

With reference to FIGS. 1 and 2, cleaning tool 10 can have first member 15 and second member 20. First member 15 and second member 20 can be constructed as a single unit as depicted in FIGS. 1 and 2, or they can be constructed as separate units that are joined together. First member 15 can define inlet 23. Inlet 23 can have any shape and size. For example, inlet 23 can be circular or square, and inlet 23 can have a width or diameter of at least 0.5 millimeter (e.g., 0.6 millimeter, 0.75 millimeter, 1.0 millimeter, 1.25 millimeters, 1.5 millimeters, or greater than 1.5 millimeters). Inlet 23 can have a circular shape with a diameter of at least one millimeter. Inlet 23 can be configured to receive a syringe (e.g., a slip fit syringe or a luer lock syringe). In another embodiment, inlet 23 can be configured to allow connection to tubing (e.g., flexible tubing that can be connect to, for example, a pump or a large syringe).

Second member 20 can be configured to fit into the magazine receiving well of a brachytherapy applicator. When positioned in a magazine receiving well, second member 20 can have a clearance of at least 0.01 millimeter on each side (e.g., 0.015 millimeter, 0.02 millimeter, 0.05 millimeter, 0.1 millimeter, 0.2 millimeter, 0.5 millimeter, one millimeter, three millimeters, or more than three millimeters).

Second member 20 can define one or more outlets (e.g., outlets 26, 28 and 30). First member 15 and second member 20 can be connected such that inlet 23 is fluidly connected to outlet 26, outlet 28, and/or outlet 30. Outlets 26, 28 and 30 can have any suitable size and shape. Outlets 26, 28 and 30 can be, for example, circular, square, or rectangular, or can be slits. Outlets 26, 28 and 30 can have widths or diameters of, for example, at least about 0.1 millimeter (e.g., 0.1 millimeter, 0.25 millimeter, 0.5 millimeter, 0.75 millimeter, one millimeter, 1.2 millimeters, 1.5 millimeters, two millimeters, or more than two millimeters). Outlets 26, 28 and 30 typically are smaller than inlet 23. Outlets 26, 28 and 30 can be positioned such that fluid forced into inlet 23 can exit cleaning tool 10 through outlets 26, 28 and 30 and be directed at specified areas of a brachytherapy applicator (e.g., the corners of a magazine receiving well, areas adjacent to or behind a magazine retaining structure, and/or regions between a chuck containing a magazine receiving well and an outer chuck). For example, an outlet (e.g., outlet 28) can be positioned such that when cleaning tool 10 is fully inserted into a magazine receiving well, outlet 28 is positioned outside the well. Fluid exiting cleaning tool 10 through outlet 28 thus can be directed at a space outside the magazine receiving well (e.g., a space between the magazine receiving well chuck and an outer chuck). Cleaning tool 10 can have any number of outlets. For example, cleaning tool 10 can have more than three outlets (e.g., three, four, five, six, seven, eight, nine, ten, or more than ten outlets). Any portion of cleaning tool 10 can define outlets.

Second member 20 can define first side surface 33 and second side surface 36, which can be substantially parallel to each other. Second member 20 also can define channel 38, which can extend between side surfaces 33 and 36. Channel 38 can have any suitable shape (e.g., circular, oval, square, or rectangular) and size (e.g., a width of less than 0.2 millimeter, 0.2 millimeter, 0.3 millimeter, 0.5 millimeter, 0.7 millimeter, 0.8 millimeter, 1.0 millimeter or more than 1.0 millimeter). Channel 38 can be configured to receive a seed-advancing stylet of a brachytherapy applicator, such that a stylet of the applicator can be extended through channel 38 when cleaning tool 10 is inserted into a magazine receiving well of a brachytherapy applicator. Cleaning tool 10 thus can be held in a fixed position within the magazine receiving well. A stylet can fit through channel 38 with a clearance between 0.01 millimeter and 3 millimeters (e.g., 0.015 millimeter, 0.05 millimeter, 0.075 millimeter, 0.1 millimeter, 0.2 millimeter, 0.3 millimeter, 0.5 millimeter, one millimeter, two millimeters, or three millimeters).

Cleaning tool 10 can have contoured (e.g., filleted, chamfered, beveled, or grooved) edges to facilitate insertion into a magazine receiving well and to allow strategic positioning of outlets to direct fluid into corners of the well. Second member 20, for example, can define grooved edges 43 and 45. Grooved edge 43 can define outlet 47, which can direct fluid that is flushed through cleaning tool 10 toward a corner of a magazine receiving well.

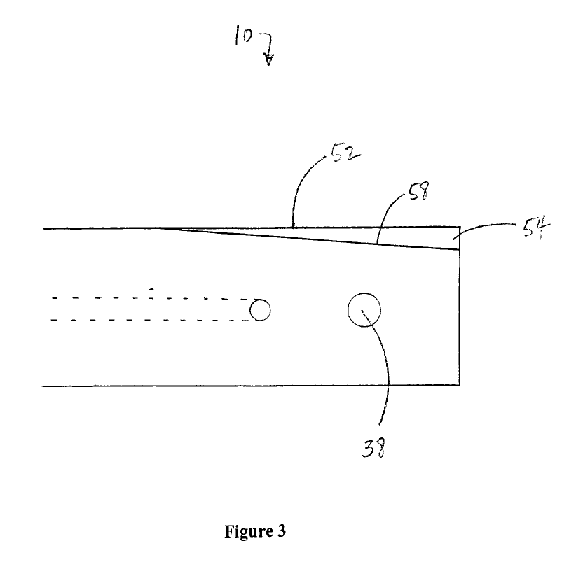

Second member 20 also can define bottom surface 50 and third side surface 52. Third side surface 52 can define recess 54, which can extend vertically along second member 20 from bottom surface 50. Recess 54 can have any suitable length. For example, the length can be more than about two millimeters (e.g., two millimeters, 2.5 millimeters, three millimeters, or more than three millimeters). Recess 54 can be configured to fit over or around a magazine retaining structure when cleaning tool 10 is inserted into a magazine receiving well. In some embodiments, recess 54 can have a depth suitable to fit over a magazine retaining structure, such as a depth between 0.3 millimeter and one millimeter (e.g., 0.3 millimeter, 0.4 millimeter, 0.5 millimeter, 0.7 millimeter, 0.9 millimeter, or one millimeter). Recess 54 can define outlet 56, which like outlets 26, 28 and 30 can be fluidly connected to inlet 23. Outlet 56 can be positioned to direct fluid toward a magazine retaining structure. With reference to FIG. 3, recess 54 can define recess surface 58, which can be sloped toward third side surface 52 such that increasing force is exerted on a magazine retaining structure as cleaning tool 10 is inserted into a magazine receiving well.

To use cleaning tool 10, a user (e.g., a clinician or a technician) can insert cleaning tool 10 into the magazine receiving well of a brachytherapy applicator and then insert a stylet of the applicator through channel 38. A syringe or other fluid delivery mechanism can be attached to inlet 23, and cleaning fluid (e.g., water, alcohol, or detergent) can be forced from the fluid delivery mechanism into cleaning device 10 through inlet 23. The cleaning fluid can exit cleaning tool 10 through outlets 26, 28, 30, 47, and 56 and thus flush different areas of a magazine receiving well.

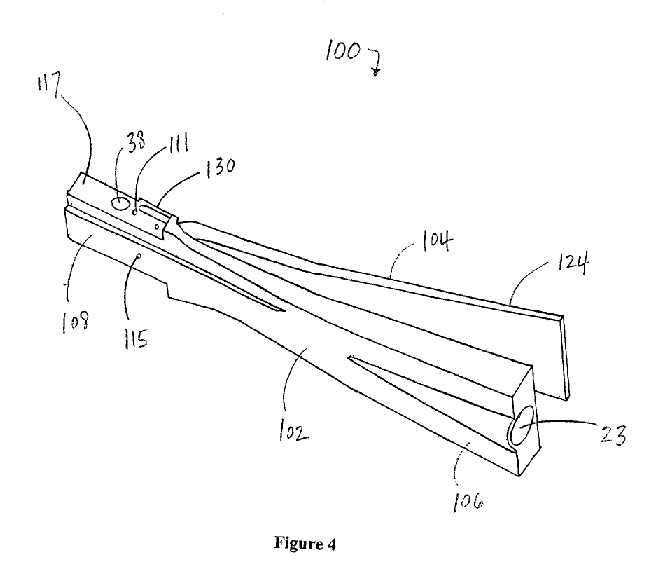

With reference to FIG. 4 through FIG. 8, cleaning tool 100 can have body 102 and pusher 104. Body 102 can have first member 106 and second member 108. As for cleaning tool 10, first member 106 can define inlet 23, while second member 108 can define at least one outlet (e.g., outlets 110, 111, 112, 113, 114, and 115) and be configured to fit into a magazine receiving well. First member 106 and second member 108 can be connected such that inlet 23 is fluidly connected to outlets 110 through 115.

Second member 108 can be configured to fit into the magazine receiving well of a brachytherapy applicator. When positioned in a magazine receiving well, second member 108 can have a clearance of at least 0.01 millimeter on each side (e.g., 0.015 millimeter, 0.02 millimeter, 0.05 millimeter, 0.1 millimeter, 0.2 millimeter, 0.5 millimeter, one millimeter, three millimeters, or more than three millimeters).

Outlets 110, 111, 112, 113, 114, and 115 can have any suitable size and shape. Outlets 110 through 115 can be, for example, circular, square, or rectangular, or can be slits. Outlets 110 through 115 can have widths or diameters of, for example, at least about 0.1 millimeter (e.g., 0.1 millimeter, 0.25 millimeter, 0.5 millimeter, 0.75 millimeter, one millimeter, 1.2 millimeters, 1.5 millimeters, two millimeters, or more than two millimeters). Outlets 110 through 115 can be positioned such that fluid forced into inlet 23 can exit cleaning tool 100 through outlets 110, 111, 112, 113, 114, and 115 and be directed at specified areas of a brachytherapy applicator (e.g., the corners of a magazine receiving well, areas adjacent to or behind a magazine retaining structure, and/or regions between a chuck containing a magazine receiving well and an outer chuck). For example, an outlet (e.g., outlet 115) can be positioned such that when cleaning tool 100 is fully inserted into a magazine receiving well, outlet 115 is positioned outside the well. Fluid exiting cleaning tool 100 through outlet 115 thus can be directed at a space outside the magazine receiving well (e.g., a space between the magazine receiving well chuck and an outer chuck). Cleaning tool 100 can have any number of outlets, For example, cleaning tool 100 can have more than three outlets (e.g., three, four, five, six, seven, eight, nine, ten, or more than ten outlets), and the outlets can be defined by any portion of cleaning tool 100.

Body 102 can define side surfaces 116 and 117, which can be substantially parallel to each other. Body 102 further can define channel 38, which can extend between side surfaces 116 and 117. In addition, body 102 can define side surface 118 and bottom surface 119. Side surface 118 can define recess 54, which can extend vertically along side surfaced 118 from bottom surface 119. Recess 54 can define outlet 56. Outlet 56 can be fluidly connected to inlet 23.

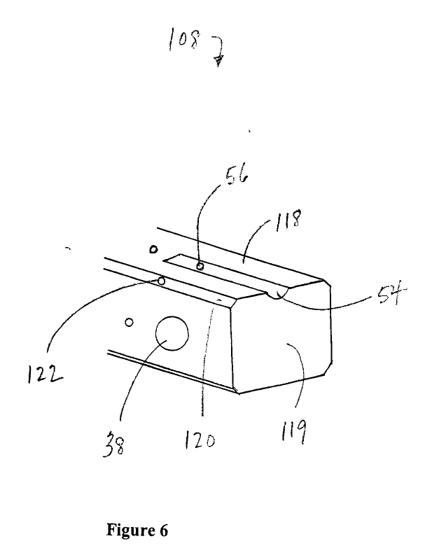

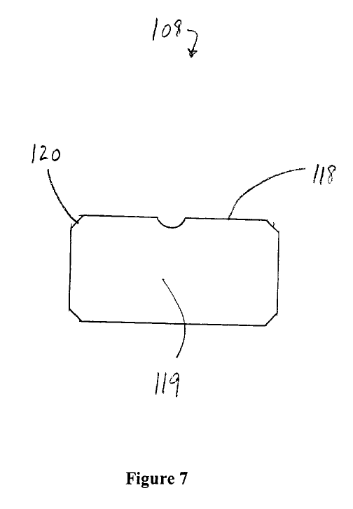

Second member 108 can have contoured (e.g., filleted, chamfered, beveled, or grooved) edges to facilitate insertion into a magazine receiving well and to allow strategic positioning of outlets to direct fluid into corners of the well. With reference to FIGS. 6 and 7, second member 108 can have beveled edge 120. Beveled edge 120 can define outlet 122, which can be fluidly connected to inlet 23. Outlet 122 can direct fluid that is flushed through cleaning tool 100 toward a corner of a magazine receiving well.

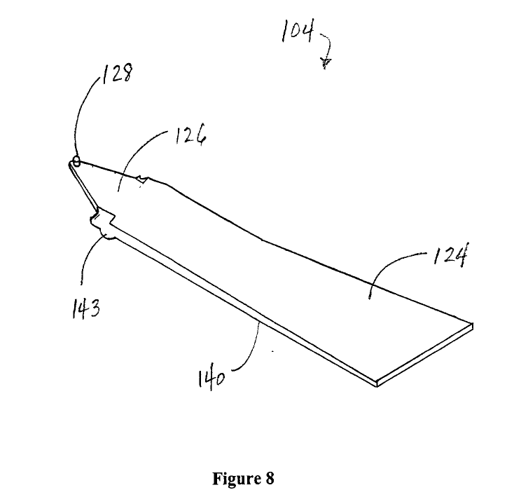

Pusher 104 can be designed to apply force to a magazine retaining structure (e.g., a ball-plunger, a leaf spring, or a Bellville spring) in a magazine receiving well. As depicted in FIG. 4 and FIG. 8, pusher 104 can define arm 124 and base 126. Base 126 can be attached to arm 124 at any suitable angle. For example, base 126 and arm 124 can be attached at an angle between 135 degrees and 180 degrees (e.g., 137 degrees, 145 degrees, 150 degrees, 155 degrees, 160 degrees, 165 degrees, 170 degrees, 175 degrees, or 179 degrees). Arm 124 and base 126 can be constructed as a single unit or can be separate units. Base 126 can define protrusion 128, which can be the portion of pusher 104 that exerts force on a magazine retaining structure. Protrusion 128 can have any suitable size and shape. For example, protrusion 128 can have a height between about 0.3 millimeter and about one millimeter (e.g., 0.3 millimeter, 0.4 millimeter, 0.5 millimeter,0.6 millimeter, 0.7 millimeter, 0.8 millimeter, 0.9 millimeter, or one millimeter).

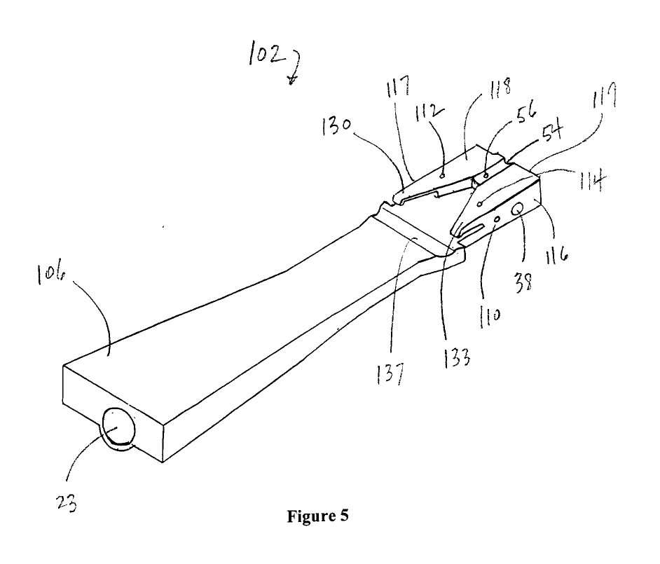

Pusher 104 can be attached to body 102 by any suitable means. As depicted in FIG. 4 and FIG. 5, body 102 can define attachment points 130 and 133. Attachment points 130 and 133 can hold pusher 104 against body 102. In some embodiments, attachment points 130 and 133 can be flexible clips or hinges (e.g., spring loaded hinges). Attachment points 130 and 133 can be configured to hold base 126 of pusher 104 against body 102, while allowing pusher 104 to move with respect to body 102. Base 126 of pusher 104 thus can be maintained in a retracted position.

Side surface 118 of body 102 can define recessed area 137. Pusher 104 can have bottom surface 140, which can define raised area 143. In some embodiments, recessed area 137 can be a pivot groove. Raised area 143 can be, for example, a rounded pivot surface. Recessed area 137 and raised area 143 can be configured to fit together, such that pusher 104 can rock back and forth against body 102 of cleaning tool 100. In other embodiments, body 102 and pusher 104 can be attached by a hinge (e.g., an integral hinge), such that pusher 104 can rock back and forth against body 102 of cleaning tool 100.

When pusher 104 is held against body 102 by attachment points 130 and 133, base 126 can be held substantially flat against second member 108, and arm 124 can be separated from first member 106 due to the angle between body 102 and arm 104. A user (e.g., a clinician or a technician) can push arm 124 against first member 106, and base 126 will move away from second member 108. When cleaning tool 100 is positioned in a magazine receiving well and a user pushes arm 124 against first member 106, protrusion 128 can exert force against a magazine retaining structure. If the magazine retaining structure is a ball-plunger, for example, protrusion 128 can actuate the ball of the ball-plunger, pushing the ball back into the ball-plunger and allowing fluid that is forced through cleaning device 100 to access the space in and around the ball-plunger. The force exerted by protrusion 128 on a ball-plunger also can actuate a ball that is stuck in the “out” position due to, for example, accumulated blood cells and debris within the ball-plunger. If the magazine retaining structure is, for example, a leaf spring or a Bellville spring that is press fit into the magazine receiving well, the force exerted by protrusion 128 also can actuate the magazine retaining structure and permit cleaning fluid to access regions adjacent to (e.g., behind) the structure.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A device for cleaning a brachytherapy applicator, wherein said device comprises:
(a) a first member defining an inlet; and
(b) a second member configured to fit into a magazine receiving well of said brachytherapy applicator, wherein said second member defines at least three outlets, and wherein said first and second members are connected such that said inlet is fluidly connected to said at least three outlets.

2. The device of claim 1, wherein said first member is configured such that a syringe fits into said inlet.

3. The device of claim 1, wherein said first member is configured such that said inlet is connectable to tubing.

4. The device of claim 1, wherein said inlet is circular with a diameter of at least about one millimeter.

5. The device of claim 1, wherein said second member defines a bottom surface and a side surface, wherein said side surface defines a recess.

6. The device of claim 5, wherein said recess extends vertically from said bottom surface for a distance greater than about two millimeters.

7. The device of claim 5, wherein the depth of said recess is between about 0.3 millimeter and about one millimeter.

8. The device of claim 5, wherein said outlet is positioned in said recess.

9. The device of claim 1, wherein said second member fits into said magazine receiving well with a clearance between about 0.01 millimeter and about three millimeters.

10. The device of claim 1, wherein said second member defines a channel.

11. The device of claim 10, wherein said second member defines two parallel side surfaces, and wherein said channel extends between said two parallel side surfaces.

12. The device of claim 10, wherein a stylet of said brachytherapy applicator fits through said channel such that said device is aligned within said magazine receiving well.

13. The device of claim 12, wherein said stylet fits through said channel with a clearance between about 0.01 millimeter and about three millimeters.

14. The device of claim 1, wherein said outlet is circular with a diameter of at least about 0.1 millimeter.

15. The device of claim 1, wherein said second member defines at least five outlets fluidly connected to said inlet.

16. The device of claim 1, wherein said first member comprises a width between about 0.5 centimeter and about two centimeters.

17. The device of claim 1, wherein said device has a length between about four centimeters and about six centimeters.

18. The device of claim 1, wherein said device comprises plastic.

19. The device of claim 1, wherein said device comprises metal.

20. A device for cleaning a brachytherapy applicator, wherein said device comprises:
(a) a first member defining an inlet;
(b) a second member configured to fit into a magazine receiving well of said brachytherapy applicator, wherein said second member defines an outlet, and wherein said first and second members are connected such that said inlet is fluidly connected to said outlet; and
(c) a pusher configured to apply force to a magazine retaining structure of said brachytherapy applicator, wherein said pusher is positioned adjacent to said outlet.

21. The device of claim 20, wherein said first member is configured such that a syringe fits into said inlet.

22. The device of claim 20, wherein said first member is configured such that said inlet is connectable to tubing.

23. The device of claim 20, wherein said inlet is circular with a diameter of at least about one millimeter.

24. The device of claim 20, wherein said second member defines a bottom surface and a side surface, wherein said side surface defines a recess.

25. The device of claim 24, wherein said recess extends vertically from said bottom surface for a distance greater than about two millimeters.

26. The device of claim 24, wherein the depth of said recess is between about 0.3 millimeter and about one millimeter.

27. The device of claim 24, wherein said outlet is positioned in said recess.

28. The device of claim 20, wherein said second member fits into said magazine receiving well with a clearance between about 0.01 millimeter and about three millimeters.

29. The device of claim 20, wherein said second member defines a channel.

30. The device of claim 29, wherein said second member defines two parallel side surfaces, and wherein said channel extends between said two parallel side surfaces.

31. The device of claim 29, wherein a stylet of said brachytherapy applicator fits through said channel such that said device is aligned within said magazine receiving well.

32. The device of claim 31, wherein said stylet fits through said channel with a clearance between about 0.01 millimeter and about three millimeters.

33. The device of claim 20, wherein said outlet is circular with a diameter of at least about 0.1 millimeter.

34. The device of claim 20, wherein said second member defines more than one outlet fluidly connected to said inlet.

35. The device of claim 20, wherein said second member defines five outlets fluidly connected to said inlet.

36. The device of claim 20, wherein said pusher comprises an arm and a base, wherein said base is attached to said arm at an angle, and wherein said base defines a protrusion.

37. The device of claim 36, wherein said angle is less than 180 degrees and greater than 135 degrees.

38. The device of claim 36, wherein the height of said protrusion is between about 0.3 millimeter and about one millimeter.

39. The device of claim 36, wherein said protrusion is positioned within about 3 millimeters of said outlet.

40. The device of claim 20, wherein said base comprises a rounded pivot surface.

41. The device of claim 40, wherein said first or second member defines a groove, and wherein said rounded pivot surface engages said groove.

42. The device of claim 41, wherein said pusher pivots about said rounded pivot surface.

43. The device of claim 42, wherein said pusher has a lever ratio between about 1/1 and about 10/1.

44. The device of claim 42, wherein said pusher has a lever ratio between about 3/1 and about 5/1.

45. The device of claim 20, wherein said first or second member comprises at least one attachment point, and wherein said pusher engages said at least one attachment point.

46. The device of claim 20, wherein said at least one attachment point is a flexible clip.

47. The device of claim 20, wherein said first member comprises a width between about 0.5 centimeter and about two centimeters.

48. The device of claim 20, wherein said device has a length between about four centimeters and about six centimeters.

49. The device of claim 20, wherein said device comprises plastic.

50. The device of claim 20, wherein said device comprises metal.

51. The device of claim 20, wherein said magazine retaining structure is a ball-plunger, and wherein said force moves the ball of said ball-plunger in a direction toward the interior of said ball-plunger.

52. A method for cleaning a brachytherapy applicator, said method comprising:
(a) providing said brachytherapy applicator, wherein said brachytherapy applicator comprises a magazine receiving well and a magazine retaining structure;
(b) providing a device for cleaning said brachytherapy applicator, wherein said device comprises:
(i) a first member defining an inlet;
(ii) a second member configured to fit into said magazine receiving well, wherein said second member defines an outlet, and wherein said first and second members are connected such that said inlet is fluidly connected to said outlet; and
(iii) a pusher configured to apply force to a said magazine retaining structure, wherein said pusher is positioned adjacent to said outlet;
(c) inserting said second member into said magazine receiving well; and
(d) inserting fluid into said inlet such that said fluid exits said outlet, thereby cleaning said brachytherapy applicator.
